(19) United States
(12) Patent Application Publication
(10) Pub. No.: US 2004/0077019 A1
(43) Pub. Date: Apr. 22, 2004

(54) Tagging compounds and process for use in aida libraries

(76) Inventor: Hubert Gstach, Vienna (AT)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/415,280

(22) PCT Filed: Oct. 29, 2001
(86) PCT No.: PCT/EP01/12483

(30) Foreign Application Priority Data
Oct. 30, 2000 (GB) 0026560.3

(51) Int. Cl. G01N 33/53; C08G 63/48; C08G 63/91

(52) U.S. Cl. 435/7.1; 525/54.1; 525/54.2

(57) ABSTRACT

A compound of formula A—B—D—X—D′—E or formula A—B—D—E—D′—X wherein A is the residue of a solid support, originating from standard materials applied in solid phase and solution phase organic chemistry, B is a linker residue having a group which allows cleavage of a compound of a formula II or a compound of formula III to liberate an D—X—D′—E, or D—E—D′—X fragment, respectively, D and D′ independently of each other are a bond or a spacer residue, E is the residue of a molecule to be investigated produced via combinatorial chemistry, and X is the residue of a fluorescent dye, which is characterized in that a compound of A—B—D—X—D′—E or of formula A—B—D—E—D′—X is tagged by at least one tag residue, and the use of such compounds in the decoding step of a screening process.

The present invention relates to tagging compounds and process, e.g. useful in fluorescence based ultra high throughput screening of encoded combinatorial compound libraries on solid support and in homogeneous solution, and in subsequent decoding of chemical structures comprised in an encoded library of compounds used in the screening process.

Combinatorial chemistry has evolved to a standard laboratory technique applied in the modem drug discovery process. A powerful and known methodology of combinatorial chemistry comprises the following steps

(a) split-and-mix synthesis on beads of a solid support (one-bead one-compound),

(b) on-bead screening of the combinatorial compound libraries with subsequent verification of the binding event(s) obtained on solid support in solution (known as AIDA-technology), and

(c) decoding the unique chemical structure causing the binding event to the target molecule(s).

Combinatorial chemistry is a useful tool for synthesis of molecules to be investigated for therapeutic use in disease states.

An appropriate method for carrying out a process of ultra high-throughput screening on solid support and in homogeneous solution by a generic labeling technology.is e.g. described in WO 00/37488. That labeling technology is based on chemically stable fluorophores, which possess reactive chemical functionality for attachment to a solid support and subsequent start of combinatorial synthesis of compound libraries. According to WO 00/37488 an at least bifunctionalized 1,3-diphenyl-1H-indazole fluorophor (generically described as AIDA-dye(s)) is attached via a cleavable linker to a solid support. The second functional group of the fluorophor is utilized for the subsequent start of library synthesis. Such inherently labelled compound libraries can be used in screening on the solid support or in homogeneous solution. The most striking chemical feature of a generic fluorescence labelled library is, that all structures constituting the compound library are covalently conjugated via a variable spacer to the fluorescent dye. The conjugates are covalently attached to the solid support via a cleavable linker. Immobilized conjugates can be used for direct screening on the solid support (on-bead screening). The fluorescent conjugates are released into solution after cleavage of the linker. Subsequent application of conventional ensemble averaging fluorescence spectroscopic techniques in assay volumes used in microtiter plates measure the affinity of the fluorescent conjugate to the target molecul in solution. In addition, single molecule spectroscopic techniques performed in microtiter volumes in so called nanocarriers can be used. The final key-step of this screening technology is the identification of the chemical structure (decoding) of the active ligand conjugated to the fluorophor-dye. Specifically according to WO 00/37488 fluorescent conjugates of formula

A—B—D—X—D′—E II

and

A—B—D—E—D′—X III

are provided, wherein

A is a solid support

B is a linker residue allowing cleavage of fluorescent conjugates of formula II or III to liberate D—X—D′—E or D—E—D′—X fragment, respectively,

X is a fluorescent dye residue, e.g. originating from a compound of formula I,

D and D′ are independently of each other a bond or a spacer residue, and

E is the residue of the molecule to be investigated, e.g. of the molecule which is prepared in the conjugates by combinatorial chemistry.

We have now surprisingly found a process which improves the decoding of a molecule to be investigated, e.g. also designated as a ligand, and which is part of such conjugates, e.g. after a binding event to a target has ocurred. The process according to the present invention includes a compound of formula II or of formula III, which compound is tagged, e.g. chemically reacted with, a tag residue, e.g. a tag residue originates from a chemical compound of low and defined molecular weight which is bound to a chemical group of which the tag residue can be splitt off during decoding, e.g. if using MS-spectrography for decoding, a tag residue can be found as a fragment. Preferably the process of the present invention includes a fluorescent dye, e.g. a compound of formula I, tagged with a tag residue, e.g. reacted with, a tag residue e.g. which tag residue includes groups, known to be able to bind to nitrogen, such as alkyl, cycloalkyl, cycloalkylalkyl, aralkyl, heterocyclylalkyl, covalently bound to nitrogen. Accordingly tagged compounds of formula I, formula II or of formula III may be used for the decoding of the molecule originating from E in a fluorescent conjugate of formula II or III, i.e. the molecule to be investigated.

In one aspect the present invention provides a compound of formula

A—B—D—X—D′—E II

or a compound of formula

A—B—D—E—D′—X III,

wherein

A is the residue of a solid support, originating from standard materials applied in solid phase and solution phase organic chemistry,

B is a linker residue having a group which allows cleavage of a compound of formula II or a compound of formula III to liberate an D—X—D′—E, or D—E—D′—X fragment, respectively,

D and D′ independently of each other are a bond or a spacer residue,

E is the residue of a molecule to be investigated produced via combinatorial chemistry, e.g. in any stage of the preparation process; and

X is the residue of a fluorescent dye,

characterized in that a compound of formula II or of formula III is tagged by at least one tag residue; e.g. in a compound of formula II or of formula III X is tagged by a tertiary amine, e.g. X is bound to a tertiary amine, which is additionally bound to a tag residue, e.g. alkyl, cycloalkyl, cycloalkylalkyl, aralkyl, heterocyclylalkyl, and to D′ (or E, if D′ is a bond), or to D (or E, if D is a bond), in a compound of formula II or of formula III.

In a compound of formula II and a compound of formula III preferably

A is the residue of a solid support, e.g. originating from a solid support known to be useful as a solid support in combinatorial, peptide and oligonucleotide chemistry, having the ability to covalently bind to B, e.g. including functionalized polystyrene based resins, polyacrylamide based polymers, polystyrene/polydimethylacrylamide composites, PEGA resins, polystyrene-polyoxyethylene based supports, Tentagel, PEG-polystyrene graft polymeric supports, glass surfaces, functionalized surfaces, materials grafted with functionalized surfaces, polyethylenglycol;

B is a linker residue e.g. originating from groups known to be useful as linkers in combinatorial, peptide and oligonucleotide chemistry, e.g. including acid labile, base labile, light labile, redox-labile, and masked linkers, e.g. benzyl, benzhydryl, benzhydryliden, trityl, xanthenyl, benzoin, silicon, or allyl based linkers, which comprise the ability to covalently bind to A and to D, if D is a spacer; or to A and to X or E, if D is a bond;

D and D′ are independently of each other a bond or the residue of a spacer, originating from groups known to be useful as spacers in combinatorial, peptide and oligonucleotide chemistry, with the ability to covalently bind to B and X or E; or to E and X, respectively; e.g. including

substituted alkanes, cycloalkanes and cycloalkenes, at least substituted by one or more, e.g. two, amino groups; or by at least one amino group and at least one hydroxy group;

substituted cycloalkylalkanes, at least substituted by one or more, e.g. two, amino groups; or by at least one amino group and at least one hydroxy group;

substituted arylalkanes, at least substituted in the alkyl part by one or more, e.g. two, amino groups; or by at least one amino group and at least one hydroxy group;

substituted alkylaromatic compounds, at least substituted in the alkyl part by one or more, e.g. two, amino groups; or by at least one amino group and at least one hydroxy group;

an aliphatic, heterocyclic ring, having 5 to 6 ring members and at least two heteroatoms selected from N, optionally anellated with another ring system; or amino acids;

for example diaminoalkanes, e.g. α,ω-diaminoalkanes; diaminocycloalkyl, such as diaminocyclohexyl; bis-(aminoalkyl)-substituted aryl, such as bis-(aminomethyl)-substituted phenyl; alkanes substituted by amino and hydroxy; such as α-amino-ω-hydroxy-alkanes; alkylamines; cyclic alkyldiamines or amino acids,

e.g. without or with additional functionality in the side chain; and said spacer residue being bound via functional groups, e.g. hydroxy, amino groups; to X and E, if D′ is a spacer; and to B and X or B and E, if D is a spacer;

E is the residue of a molecule to be investigated and produced via combinatorial chemistry, e.g. In any stage of the preparation process, e.g. originating from a low molecular weight compound, having the ability to covalently bind to D′ or to X, if D′ is a bond; or to D or B (if D is a bond) and to D′ or X (if D′ is a bond); e.g. Including a carbohydrate, with functional groups, such as an aliphatic, aromatic and/or heterocyclic compound, e.g. with all kind of chemical functionality; and

X is the residue of a compound of formula

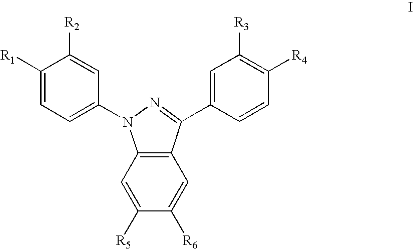

wherein

one of R₁ and R₂ and one of R₃ and R₄ is hydrogen, and the other R₁ or R₂; and R₃ or R₄ is independently of each other

—COOH, —COOR₇, —CONHR_{tag}, —CONR_{tag}(CH₂)ₙOH, —CONR₈R₉, —CH₂OH, —CH₂NHR_{tag}, —NO₂, —NR₁₀R₁₁, —NR_{tag}COR₁₂, Cl, Br, F, —CF₃, alkoxy, e.g. including unsubstituted alkoxy and substituted alkoxy, e.g. substituted in the alkyl part by aryl, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂)ₙNR_{tag}H, unsubstituted or substituted alkyl, e.g. alkyl substituted, e.g. preferably at the terminal carbon atom, by

—COOH, —COOR₇, —CONR_{tag}H, —CONR₈R₉, —CONR_{tag}(CH₂)ₙOH, —CH₂OH, —CH₂NH₂, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂)ₙNR_{tag}H;

R₅ and R₆ are hydrogen, or one of R₅ and R₆ is hydrogen and the other is hydrogen, halogen, unsubstituted alkoxy, substituted alkoxy, e.g. substituted by

-aryl, —NO₂, —NR₁₀R₁₁, —NR_{tag}COR₁₂;

unsubstituted alkyl or substituted alkyl, e.g. substituted, e.g. preferably at the terminal carbon atom, by

—COOH, —COOR₇, —CONHR_{tag, —CONR}₈R₉, —CONR_{tag}(CH₂)ₙOH, —CH₂OH, —CH₂NHR_{tag}, —N═C═O, —N═C═S, —SO₃H, —SO₂NR_{tag}(CH₂)ₙNH₂, —SO₂NH(CH₂)ₙNHR_{tag}, —CONR_{tag}(CH₂)ₙNH₂, —CONH(CH₂)ₙNHR_{tag};

n=2to 8,

R₇ is a carboxyl-protecting or carboxyl-activating group;

R₈ and R₉ together with the nitrogen atom to which they are attached form heterocyclyl, with the proviso that piperazine is excluded;

R₁₀ and R₁₁ are independently of each other hydrogen or R_{tag},

R₁₂ is alkyl, aryl, aralkyl, unprotected or protected amino or halogen;

R_{tag} is hydrogen, alkyl, cycloalkyl, cycloalkylalkyl, aralkyl, heterocyclylalkyl with the proviso that unsubstituted methyl and unsubstituted cyclopropylmethyl are excluded;

with the proviso that in a compound of formula I

at least one R_{tag} is present,

at least one R_{tag} is other than hydrogen. and

at least one, preferably two, functional groups are present in the meanings of R₁, R₂, R₃, R₄, R₅, and R₆ which have the ability to covalently bind to one or two further reactants.

The “ability to covalently bind” as used herein means, that at least one, preferably two functional groups are present which enable (easily) a chemical reaction with either one or two reaction partners, e.g. such reaction partners from which residues A, B, D, D′ and X in a compound of formula II and of formula III are derived.

If not otherwise defined herein alkyl or alkane includes (C₁₋₂₂)alkyl or alkane. e.g. (C₁₋₈)alkyl or alkane, such as (C₁₋₄)alkyl or alkane. Cycloalkyl, cycloalkane or cycloalkene includes (C₃₋₇)cycloalkyl, cycloalkane or cycloalkene, such as (C₅₋₆)cycloalkyl, cycloalkane or cycloalkene, e.g. anellated with another ring (system). Alkoxy includes (C₁₋₁₈)alkoxy, such as (C₁₋₃)alkoxy, e.g. (C₁₋₄)alkoxy. Aryl or aromatic compound includes (C₅₋₁₈)aryl or aromatic compound, e.g. phenyl or benzene, e.g. anellated with another ring (system). A carboxyl protecting or carboxyl activating group include appropriate protecting or activating groups, e.g. groups as conventional in organic chemistry, such as groups which can be (easily) split off; or groups which enables (easily) further reaction, respectively. Heterocyclyl includes a ring (system) having 5 to 7, preferably 5 to 6 ring members and 1 to 4 heteroatoms, e.g. selected from N, O, S; e.g. anellated with another ring (system). Any group mentioned herein may be unsubstituted or substituted, e.g. substituted by groups that are conventional in organic chemistry, preferably, if not otherwise defined herein, chemically inert groups. Amino protection groups include appropriate amino protecting groups, e.g. protecting groups as conventional in organic chemistry, e.g. such as tert-butyloxycarbonyl (BOC), 9-fluorenylmethoxycarbonyl (Fmoc), phthalimido, trifluoromethylcarbonyl, methoxycarbonyl, ethoxycarbonyl, benzyloxycarbonyl, allyloxycarbonyl, 2,2,2-trichloroethoxycarbonyl, 2-(trimethylsilyl)ethoxycarbonyl.

Preferred compounds of formula II and formula III include compounds of formula II and formula III wherein

A, B, D, D′ and E are as defined above, and X is the residue of a compound of formula I, wherein one of R₁ and R₂ is hydrogen and the other is a group —CONR_{tag}, one of the groups R₃ and R₄ is hydrogen and the other is a group —CONR_{tag}, R₅ and R₆ are hydrogen; and R_{tag} is as defined above.

In another aspect the present invention provides a compound of formula

D—X—D′—E IV, or

D—E—D′—X V, or

E—D′—X VI

wherein E, and X are as defined above and D and D′ independently of each other are the residue of a spacer, or are a bond, or ar not present;

e.g. X is the residue of a compound of formula I,

characterized in that a compound of formula IV or of formula V or of formula VI is tagged by at least one tag residue.

Preferably in a compound of formulae IV, V or VI, the residue X is a residue of a compound of formula I, wherein

at least one R_{tag} is present, and

at least one R_{tag} is other than hydrogen; and at least one of the residues R₁, R₂, R₃, R₄, R₅, and R₆ is bound to either D, D′ or E.

Compounds of formula I wherein at least one of R₁, R₂, R₃, R₄, R5, and R₆ comprises a group —N—R_{tag} are novel.

In another aspect the present invention provides a compound of formula I, wherein

one of R₁ and R₂ and one of R₃ and R₄ is hydrogen, and the other R₁ or R₂; and R₃ or R₄ is independently of each other

—COOH, —COOR₇, —CONHR_{tag}, —CONR_{tag}(CH₂)ₙOH, —CONR₈R₉, —CH₂OH, —CH₂NHR_{tag}, —NO₂, —NR₁₀OR₁₁, —NR_{tag}COR₁₂, Cl, Br, F, —CF₃, alkoxy, e.g. including unsubstituted alkoxy and substituted alkoxy, e.g. substituted in the alkyl part by aryl, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂₎ₙNRₜₐH, unsubstituted or substituted alkyl, e.g. alkyl substituted, e.g. preferably at the terminal carbon atom, by

—COOH, —COOR₇, —CONR_{tag}H, —CONR₈R₉, —CONR_{tag}(CH₂)ₙOH, —CH₂OH, —CH₂NH₂, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂)ₙNR_{tag}H;

R₅ and R₆ are hydrogen, or one of R₅ and R₆ is hydrogen and the other is hydrogen, halogen, unsubstituted alkoxy, substituted alkoxy, e.g. substituted by

-aryl, —NO₂, —NR₁₀R₁₁, —NR_{tag}COR₁₂;

unsubstituted alkyl or substituted alkyl, e.g. substituted, e.g. preferably at the terminal carbon atom, by

—COOH, —COOR₇, —CONHR_{tag}, —CONR₈R₉, —CONR_{tag}(CH₂)ₙOH, —CH₂OH, —CH₂NHR_{tag}, —N═C═O, —N═C═S, —SO₃H, —SO₂NR_{tag}(CH₂)ₙNH₂, —SO₂NH(CH₂)ₙNHR_{tag}, —CONR_{tag}(CH₂)ₙNH₂, —CONH(CH₂)ₙNHR_{tag};

n=2 to 8,

R₇ is a carboxyl-protecting or carboxyl-activating group

R₈ and R₉ together with the nitrogen atom to which they are attached form heterocyclyl, with the proviso that piperazine is excluded;

R₁₀ and R₁₁ are independently of each other hydrogen or R_{tag},

R₁₂ is alkyl, aryl, aralkyl, unprotected or protected amino or halogen;

R_{tag} is hydrogen, alkyl, cycloalkyl, cycloalkylalkyl, aralkyl, heterocyclylalkyl, with the proviso that unsubstituted methyl and unsubstituted cyclopropylmethyl are excluded;

with the proviso that in a compound of formula I

at least one R_{tag} is present,

at least one R_{tag} is other than hydrogen. and

at least one, preferably two, functional groups are present in the meanings of R₁, R₂, R₃, R₄, R₅, and R₆ which have the ability to covalently bind to one or two further reactants.

Preferably R_{tag} includes unsubstituted alkyl, with the proviso that methyl is excluded, e.g. (C₂₋₂₂)alkyl, such as (C₂₋₆)alkyl, e.g. 3,3-dimethylbutyl, and alkyl substituted by

cycloalkyl, e.g. (C3-7)cycloalkyl, such as cyclohexylmethyl, with the proviso that 2-cyclopropylethyl is excluded,

aryl, e.g. including phenyl, such as 4-methylphenylmethyl phenyl, 4-chlorophenylmethyl, 4-bromophenylmethyl, 4-trifluoromethylphenylmethyl, 4-methoxyphenylmethyl, 2-(2-chlorophenyl)ethyl, 2-(3,4-dimethoxyphenyl)methyl, 3-fluorophenylmethyl, 2-(4-methoxyphenyl)ethyl, 3-phenylpropyl,

alkoxy, e.g. including (C₁₋₄)alkoxy, such as 2-methoxyethyl, 2-ethoxyethyl, 2-propoxyethyl, 2-isopropoxyethyl, 3-methoxypropyl, 3-ethoxypropyl, 3-propoxypropyl, 3-isopropoxypropyl, 3-butoxypropyl, 3-isobutoxypropyl, 3(2-ethyl-hexoxy)propyl, 3-hexoxypropyl, 4-hexoybutyl,

alkoxyalkoxy, such as 2-[2-(methoxy)ethoxy]ethyl, 2-[2-(dodecyloxy)ethoxy]ethyl, 3-[2-(methoxy)ethoxy]propyl, 3-[2-(ethox)ethoxy]propyl,

alkoxyalkoxyalkoxy, such as hexoxyethoxyethoxyethyl,

hydroxyalkoxy, such as 2-(2-hydroxyethoxy)ethyl,

aminoalkoxy, aminoalkoxyalkoxy, aminoalkoxyalkoxyalkoxy, e.g. wherein the amine group is unprotected or protected; such as 2-aminoethoxymethyl, 2-(2-aminoethoxy)ethyl, 3-(2-aminoethoxy)propyl, 2-(2-N-Fmoc-aminoethoxy)ethyl, 3-(2-dimethylaminoethoxy)propyl, 2-(2-aminoethoxy)ethoxyethyl, 3-(3-aminopropoxybutoxypropyl, 3-(3-aminopropoxy)ethoxyethoxypropyl;

arylalkoxy, e.g. benzyloxy,

aryloxy, e.g. including phenyloxy, such as 4-methoxyphenyloxymethyl, 3,4-dimethoxyphenyloxymethyl, 3,4,5-trimethoxyphenyloxymethyl, 2-phenyloxyethyl, 2-(4-methoxyphenyloxy)ethyl, 2-(3,4-dimethoxyphenyloxy)ethyl, 2-(2-methoxyphenyloxy)ethyl, 2-(4-methoxyphenyloxy)ethyl, 2-chlorophenyloxymethyl,2-(3,4,5-trimethoxyphenyloxy)ethyl, 2-(2-chlorophenyloxy)ethyl, 2-(3-trifluorophenyloxy)ethyl, 2-(4-trifluorophenyloxy)ethyl, 3-(4-acetoxyaminophenyloxy)propyl, 4-phenylbutyl,

hetrocyclyloxy, e.g. including (4-amino-furazan-5yl)oxy,

heterocyclyl, e.g. including 4-pyridylmethyl, 2-(2-thienyl)ethyl, 2-morpholinoethyl; 3-(2-oxopyrrolidin-1yl)-propyl,

amino, e.g. unprotected amino and protected amino, such as 2-aminoethyl, 3-aminopropyl, 2-N-acetoxycarbonyl-aminoethyl, 2-(4-methoxyphenyl)carbonylaminoethyl, 3-N-acetoxycarbonyl-aminopropyl,

cycloalkyl, e.g. (C₃₋₇)cycloalkyl, such as cyclopentyl

Compounds provided by the present invention, e.g. including a compound of formula I, II, III, IV, V and VI, are hereinafter designated as compounds of the present invention.

A compound of the present invention is useful in the decoding step in a chemical combinatorial screening process. This decoding step according to the present invention is facilitated due to the presence of a nitrogen atom in a compound according to the present invention which is substituted, i.e. partial-encoded, by a group R_{tag}.

In another aspect the present invention provides the use of a compound of the present invention in the decoding step of a screening process.

Compounds of the present invention may be produced as appropriate, e.g. according to methods as conventional, e.g. or according as described herein.

In another aspect the present invention provides a process for the synthesis of generic fluorescence labelled libraries (AIDA-libraries) on a conventionally used linker cleavable by light wherein the first combinatorial step of fluorescence labelled libraries is partial-encoded comprising providing, e.g. and further reacting according to Scheme 1, a set of compounds bound to at least to one tag residue, e.g. to R_{tag}, which set of compounds consists in compounds of the present invention wherein the tag residues, e.g. R_{tag}, have different molecular weights.

A linker cleavable by light is also known as a photolinker. That process according to the present invention e.g. may be carried out according to the following reaction Scheme 1

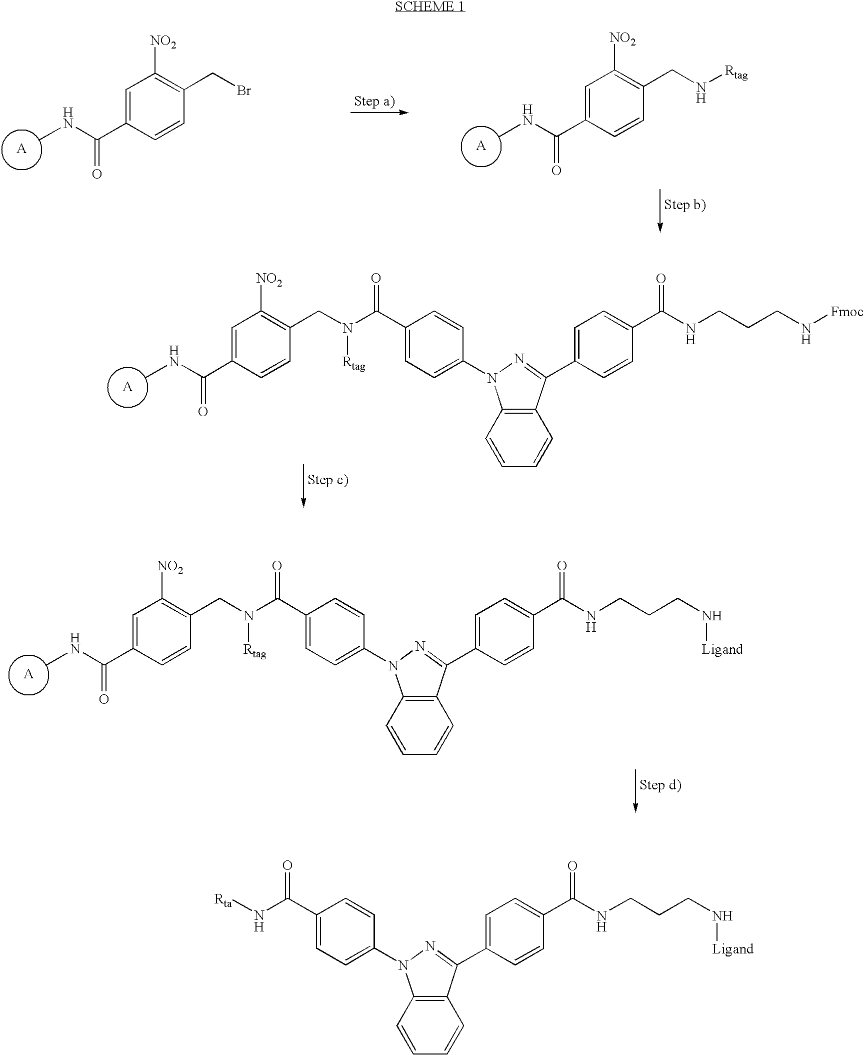

The synthesis of a fluorescence labelled AIDA-library on a photolabile 4-bromomethyl-3-nitro-benzoic acid linker which is fixed on a solid support A (e.g. a resin bead) may e.g. be started by nucleophilic substitution of the benzylic bromide by a primary amine of formula HNR_{tag} (Step a)), followed by coupling with an, e.g. Fmoc-protected AIDA-dye which is e.g. known from WO 00/37488 (Step b)); and ligand reaction (Step c)) with the compound to be investigated, and cleavage by light (Step d)). The fluorophor conjugated library compounds are released into solution as amides. The amide function on the 4 position of the 1-phenylsubstituent of the AIDA molecule is not seen as an int gral part of the synthesized library compound, as it is also not th case for the fluorophor (AIDA) itself. The distance between amide functionality and attachment point of the ligands is >20 Angstroem. Consequently, the terminal amide functionality of the AIDA-conjugates is a variable element having most likely little influence on the conformations adopted by the synthesized library compound.

Steps a) to d) may be carried out according to a method as appropriate, e.g. according to a method as conventional. A set of compounds is obtained wherein the first combinatorial step of fluorescence labelled libraries is encoded by the specific R_{tag} used.

In another aspect the present invention provides a process for the synthesis of generic fluorescence labelled libraries (AIDA-libraries) on a conventionally used linker cleavable by acid; wherein the first combinatorial step of fluorescence labelled libraries is partial-encoded, comprising providing a set of compounds of the present invention covalently bound to at least one tag residue, e.g. R_{tag}, which set of compounds consists in compounds as claimed in any one of claims 1 to 3, wherein the tag residues, e.g. R_{tag}, have different molecular weights.

A conventionally used linker cleavable by strong acid is e.g. known as Rink linker. That process according to the present invention e.g. may be carried out according to a method as conventional, or, e.g. as follows:

The (partial)-encoding of the first combinatorial step of fluorescence labelled libraries on the acid labile Rink Amide (RAM) linker follows a different process compared to the corresponding photolinker process. A direct transformation of the primary amine of the RAM-linker into a tagged secondary amine may e.g. require a reductive alkylation step with a set of tagged aldehydes of formula R_{tag}—CHO, in which set the molecular weight of the different aldehydes is different prior to the coupling of the fluorophor, see e.g. Brown, E. G. et al, Tetrahedron Letters (1997), 38(49), p. 8457-8460 (Step a)). A different process which may be used involves reacting the deprotected Rink Amide resin with bromo acetic acid, followed by nucleophilic substitution with a set of tagged primary amines of formula R_{tag}—NH₂, in which set the molecular weight of the different amines is different (Step a1) and Step a2)). The resulting secondary amine is coupled to the fluorophor reagent Fmoc-AIDA-OH (Step b). The first combinatorial step (ligand reaction—Step d)) is started after Fmoc-AIDA deprotection (Step c). Acidic cleavage (Step e)) with acids, e.g. strong acids, such as trifluoroacetic acid, of the linker results in amides or N-(carbamoylmethyl) substituted amides of the fluorophor-conjugated ligands, e.g. as shown in Scheme 2 below.

The reactions described in Steps a), a1), a2), b), c) and d) are known per se and may be carried out according, e.g. analogously, to, a method as conventional.

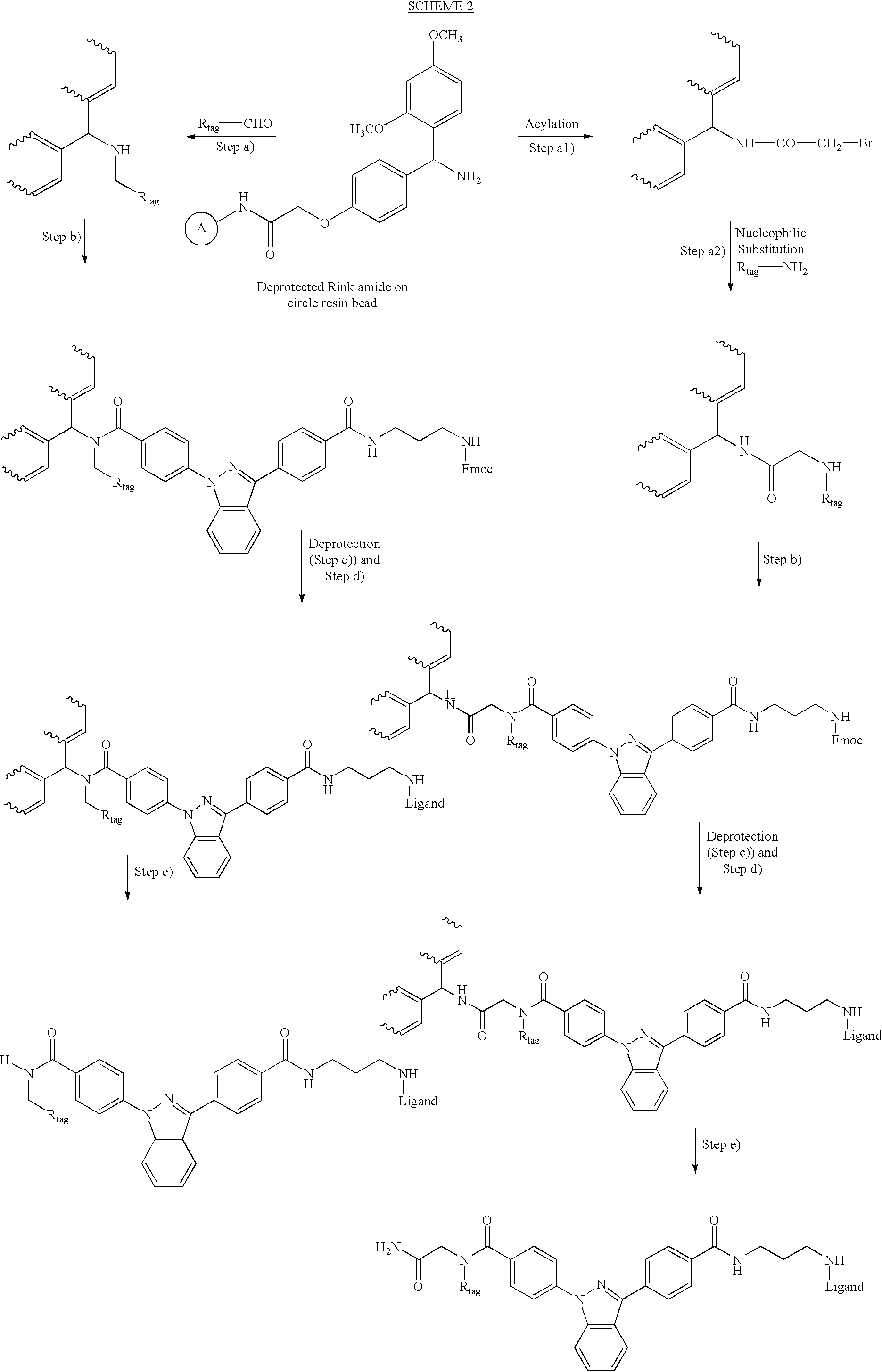

In another aspect the present invention provides a process for the decoding of building blocks of generic fluorescence labelled libraries (AIDA-libraries) comprising reading a tag residue, e.g. R_{tag}, in a compound of formula I, IV, V, or VI according to the present invention by use of MS-spectrography.

The tag-reading for decoding of the first combinatorial step of a generic fluorescence labelled library (AIDA-library) may be performed with LC/MS, e.g. of the cleaved fluorophor-conjugated ligand molecules, e.g. such as shown below in Scheme 3 (Ligand=E in a compound of formula IV, V or VI). The mass spectra contain the information about the molecular weight (MS in ESI⁺: [MH]⁺) of such conjugates. The knowledge about the molecular weight of the fluorophor-conjugated ligand is in many cases not sufficient for decoding the structure, because in small-molecule libraries some of the compounds may coincidentally have the same nominal mass. In such a case the necessary information about the structure of the fluorophors is obtained from the fragmented molecular-ion ([MH]⁺) in the mass spectrometer which indicates the molecular weight ot the R_{tag} used. The conjugates contain amide bonds which are preferred starting points of fragmentation after ionization. A couple of such fragment-ions carry the full information about the tag on the AIDA-fluorophor. The desired information becomes visible several times in the mass spectrum of the tagged fluorophor-conjugate; e.g. exemplified fragments of the AIDA-fluorophore are shown in Scheme 3 below. MS/MS (CDI: collision induced dissociation) is used to verify fragment-ions, e.g. as shown in Scheme 3, by their molecular-ion [MH]⁺.

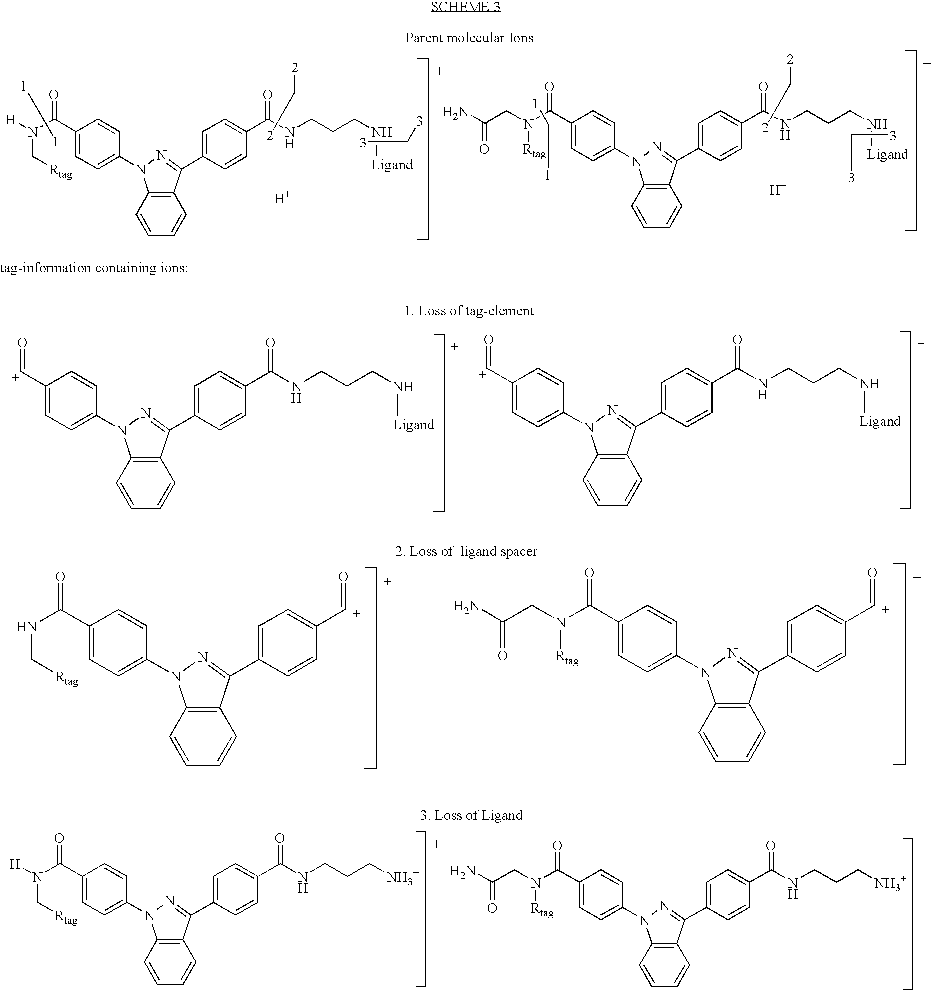

In another aspect the present invention provides a process for the decoding of building blocks obtained by more than one combinatorial step of generic fluorescence labelled libraries (AIDA-libraries) comprising a split-and-mix-and-divide production of generically fluorescence (AIDA) labelled compound libraries in more than one combinatorial step, e.g. up to 4, and reading a tag residue, e.g. R_{tag}, in a compound of formula I, IV, V, or VI according to the present invention by use of MS-spectrography.

Partial-encoding of the 1^{st} combinatorial step is not only useful in the production of small AIDA-labelled libraries. Fluorophor-tagging by a tag residue according to the present invention in combination with, e.g. known, deconvolution strategies of combinatorial chemistry creates also a valulable tool for decoding of even larger libraries. This aspect is outlined in a split-and-mix-and-divide production scheme for (e.g. on-bead) libraries synthesized in four combinatorial steps as exemplified in Scheme 4 below.

Three consecutive combinatorial steps (R¹×R²×R³) substantiate mother-libraries, which serve as the source for daughter-libraries (R¹×R²×R³×R⁴) created in a further 4^{th} combinatorial step. Non-combinatorial steps may also be carried out, e.g. in order to achieve functional group transformations for the purpose of added diversity.

The building blocks obtained in the 2^{nd} combinatorial step (R²) are selected in a way that the building blocks obtained have different molecular weights by use of reactants having different molecular weights. The resulting tagged-AIDA conjugated-building blocks of the 3^{rd} combinatorial step (R³) are kept in separate vials (no mix); thus, R³ needs no decoding. The number of building blocks used in the 3^{rd} combinatorial step may define the number of sub-libraries of the entire library. A library obtained in the 3^{rd} combinatorial step is divided into a set of aliquots; the number of aliquots may be defined by the number of building blocks intended to be used in the 4^{th} combinatorial step (R⁴) (libraries-from-libraries step). R⁴ produces daughter-libraries of equal architecture compared to the mother-library which are kept separately; thus R⁴ needs no decoding. The mother-library and all daughter-libraries derived thereof may contain the same number of sub-libraries, and each sub may contain equal numbers of compounds. Tagging the first reaction step, no mass-isobars in R², separately kept sub-libraries R³, and subsequent dividing in R⁴ enable the decoding of the structures even if in the case that all structures comprised in the library obtained after the last combinatorial step would coincidentally have the same molecular weight by molecular-ion ([MH]⁺) and tag-information (as fragment-ions derived from [MH]⁺, see e.g. Scheme 3 above) e.g. which are present in the mass spectrum data obtained from the material cleaved from a single bead.

In another aspect the present invention provides a process for the decoding of building blocks of generic fluorescence labelled libraries (AIDA-libraries) comprising

a) tagging a first combinatorial step with a tag residue, e.g. R_{tag}, e.g. on bead, and carrying out the first combinatorial step, in different vials for each building block expected to be obtained,

b) mixing the content of the vials containing the different building blocks obtained in the first combinatorial step,

c) carrying out a second combinatorial step such, that the building blocks obtained after the second combinatorial step have different molecular weights, e.g. by selecting and using reactants having different molecular weights, in a mixture obtained in b), for each building block in separate vials, and, if desired,

d) mixing the content of the vials obtained in step c) and carrying out a third combinatorial step, for each building block in different vials, and if desired,

e) carrying out a fourth combinatorial step for each building block in at least one, preferably in each, of the vials obtained in step d), and

f) reading a tag residue, e.g. R_{tag}, in a compound of formula I, IV, V, or VI according to the present invention by use of MS-spectrography, e.g. MS/MS (collision induced dissociation=CID).

Steps c) and d) are not necessarily to be carried out, e.g. if only two combinatorial steps are desired step c) and step d) can be omitted.

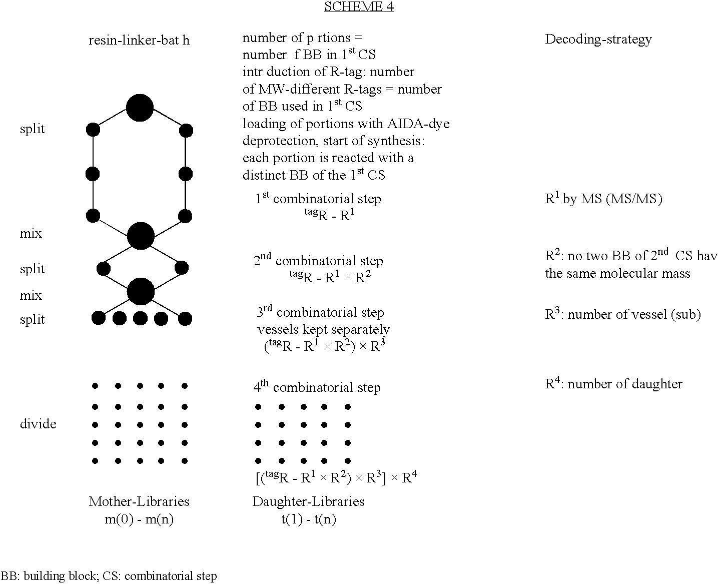

Tagging according to the present invention may not only be useful for decoding, e.g. as described above, but e.g. may be additionally used to modify the physico-chemical properties of generic fluorescence labelled conjugated ligands, e.g. AIDA conjugated ligands, such as of formula IV, V and VI, e.g. by selecting such tag residues which influences the physico chemical properties in a generic fluorescence labelled conjugated ligand, e.g. acompound of the present invention, e.g. which change the physico-chemical properties of a generic fluorescence labelled conjugated ligand, e.g. of a compound of the present invention, with respect to a non-tagged and otherwise identical compound.

In another aspect the present invention provides the use of a compound comprising a tag residue according to the present invention in the modification of physio-chemical properties of generic fluorescence labelled conjugated ligands, e.g. AIDA-conjugated ligands.

An important application of such use includes e.g. the improvement of the solubility of generic fluorescence labelled conjugated ligand, e.g. AIDA-conjugated ligands. As already described, a positive on-bead binding event of a target to an AIDA-conjugated ligand on a resin bead can subsequently be verified (determined) in solution after cleavage of the conjugate from the isolated bead (e.g. corresponding to AIDA-technology). Thus, improvement of solubility of a generic fluorescence labelled conjugated ligand in the solvent system used, e.g. in an aqueous buffer system, may often be useful. Especially, the 1,3-diphenyl-1H-indazole core of the AIDA-fluorophore is a hydrophobic moiety due to the high content of aromatic rings making the AIDA-conjugated ligands less soluble in water compared to the unconjugated congeners. According to the present invention the set of the tag residues, e.g. including R_{tag}-amines, R_{tag}-aldehydes, is created by selecting such R_{tag} residues which improve solubility of a tagged compound of the present invention compared with a non-tagged, but otherwise identical compound.

In another aspect the present invention provides the use of a compound comprising a tag residue according to the present invention in the improvement of the solubility of generic fluorescence labelled conjugated ligands, e.g. AIDA-conjugated ligands, in a solvent (system), e.g. including non-aqueous and aqueous solvent (system).

We have used as a characterization of the solubilty of a generic fluorescence labelled conjugated ligand, e.g. an AIDA-conjugated ligand, its calculated logarithm of the 1-octanol/water partition coefficient (clogP-value), see e.g. Ghose, A. K., et al. J. Phys. Chem. A. (1998), 102(21), p. 3762-3772, o Chou, J. T., et al, J. Chem. Inf. Comput. Sci. (1979), 19(3), p. 172-178. In TABLE 1 below the modification of solubility in water of an ADIA dye-conjugate (having a dipeptide as a Ligand) depending on different tag residues is demonstrated. A lower clogP means better solubility in water.

TABLE 1

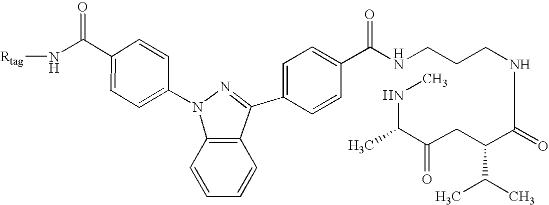

| R_{tag} | clogP |
|---|---|
| H | 2.732 |
| CH₃CH₂CH₂— | 4.004 |
| CH₃CH₂CH₂CH₂— | 4.533 |
| CH₃OCH₂CH₂— | 3.002 |
| CH₃OCH₂CH₂OCH₂CH₂— | 3.067 |
| HO—CH₂CH₂OCH₂CH₂— | 2.444 |

The clogP values in TABLE 1 are calcutated by using the CLOGP3-algorithm of BioByte, CA, US.

Combinatorial chemistry is a useful tool for synthesis of molecules to be investigated for therapeutic use in disease states. Compounds of formula II and III may e.g. be used for on bead screening of proteins, e.g. proteins known to influence disease states, e.g. in a mammal. A compound of formula II or III which is found to bind to such a protein may be a valuable pharmaceutical.

In the following examples all temperatures are in degree Celsius.

The following abbreviations are used:

TFA: trifloroacetic acid

CID: collision induced dissociation

### EXPERIMENTAL

The compounds 1 to 26 described in TABLE 2 below are compounds of formula E1, as shown below; and the compounds of examples 27 to 30 are compounds of formula E2 as shown below, wherein in compounds 1 to 3, 5, 8, 9, 15, 24, 26, 27 and 29 R_{E} is ALA (β-alaninoyl); in compounds 4, 6, 7, 10 to 14, 16 to 23, 25, 28 and 30 R_{E} is LALA (L-alaninoyl). The compounds of TABLE 2 may be prepared analogously as described in WO00/37488 but additionally introducing R_{tag} analogously as described herein.

TABLE 2

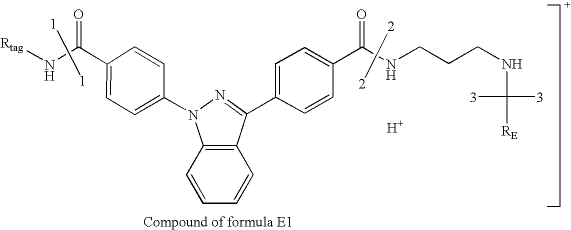

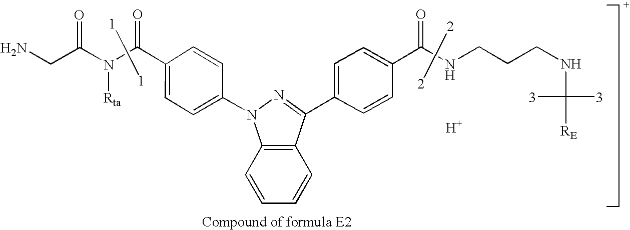

| | | Full MS* | MS/MS |
|---|---|---|---|
| R_{tag} | Ex | [MH]⁺ | Fragmentation of [MH]⁺ |
| Methyl | 1 | 499 | 1: 468 (6); 2: 354 (70); 3: 428 (10) |
| 3,3-Dimethylbutyl | 1 | 569 | 1: 468 (10); 2: 424 (70); 3: 498 (30) |
| Cyclopentyl | 3 | 553 | 1: 468 (28); 2: 408 (100); 3: 482 (35) |
| Cyclohexylmethyl | 4 | 581 | 1: 468 (20); 2: 436 (40); 3: 510 (100) |
| 3-Phenylpropyl | 5 | 603 | 1: 468 (50); 2: 458 (90); 3: 532 (10) |
| 4-Phenylbutyl | 6 | 617 | 1: 468 (15); 2: 472 (85); 3: 546 (100) |
| 2-(4-Methoxyphenyl)-ethyl | 7 | 619 | 1: 468 (15); 2: 474 (40); 3: 548 (100) |
| 4-Methylphenyl-methyl | 8 | 589 | 1: 468(20); 2: 444 (95); 3: 518 (100) |
| 4-Chlorophenyl-methyl | 9 | 609 | 1: 468 (15); 2: 464 (80); 3: 538 (30) |
| 4-Bromophenyl-methyl | 10 | 653 | 1: 468 (3); 2: 508 (45); 3: 582 (80) |
| 4-Trifluoromethyl-phenyl-methyl | 11 | 643 | 1: 468 (20); 2: 498 (40); 3: 572 (100) |
| 4-Methoxyphenyl-methyl | 12 | 605 | 1: 468 (30); 2: 460 (100); 3: 534 (80) |
| 4-Pyridylmethyl | 13 | 576 | 1: 468 (36); 2: 431 (10); 3: 505 (60) |
| 2-Methoxyethyl | 14 | 543 | 1: 468 (40); 2: 398 (56); 3: 472 (100) |
| 3-Methoxypropyl | 15 | 557 | 1: 468(98); 2: 412 (100); 3: 486 (38) |
| 3-(2-Oxopyrrolidin-1-yl)-propyl | 16 | 610 | 1: 468 (45); 2: n.d.; 3: 539 (35) |
| 2-(2-Thienyl)-ethyl | 17 | 595 | 1: 468 (20); 2: 450 (85); 3: 524 (95) |
| 2-(2-Chlorophenyl)-ethyl | 18 | 623 | 1: 468(20); 2: 478 (100); 3: 552 (65) |
| 2-Morpholino-ethyl | 19 | 598 | 1: 468 (13); 2: 453 (8); 3: 527 (40) |
| 3,3-Dimethylbutyl | 20 | 569 | 1: 468 (15); 2: 424 (75); 3: 498 (100) |
| 2-Methylpropyl | 21 | 541 | 1 and 3: 468 (20); 2: 396 (75) |
| Cyclopropylmethyl | 22 | 539 | 1 and 3: 468 (55); 2: 394 (45) |
| Ethyl | 23 | 513 | 1: 468 (15); 2: 368 (45); 3: 442 (100) |
| 2-(3,4-Dimethoxyphenyl)-methyl | 24 | 649 | 1: 468 (40); 2: 504 (100); 3: 578 (85) |
| 2-(4-Methoxyphenyl)-ethyl | 25 | 619 | 1: 468 (90); 2: 474 (100); 3: 548 (15) |
| 3-Fluorophenyl-methyl | 26 | 593 | 1: 468 (7); 2: 448 (60); 3: 522 (40) |

| | | |
|---|---|---|
| | | Full MS* |
| 2-Methoxyethyl | 27 | 600 (100, [MH]⁺);1: 468(85);2: 455 (4); 3: 529 (1) |
| 2-Methoxyethyl | 28 | 600 (100, [MH]⁺);1: 468(60);2: 455 (5);3: 529 (2) |
| 3-Methoxypropyl | 29 | 614 (100, [MH]⁺);1: 468(45);2: 469 (1 4);3: 543 (1) |
| 3-Methoxypropyl | 30 | 614 (100, [MH]⁺);1: 468(40);2: 469 (17);3: 543 (1) |

The numbers in bold (1, 2 and 3) refer to the cleavage site shown in a compound of formula E1 and of formula E2.

### METHODS FOR ANALYSIS

TentaGel resins (TentaGel S NH₂; TentaGel S RAM; bead size: 90 μm; load: 0.23-0.28 mmol/g resin) used are known and e.g. available from Rapp Polymere, Tübingen, DE. The mass spectra of the crude products may be determined on a Finnigan Thermo Quest Navigator LC/MS coupled to a Hewlett-Packard Series 1100 HPLC system. The mass spectrometer may be operated as an open access MS running under (ESI⁺/ESI⁻)- or (APCI⁺/APCI⁻)-mode. Samples may be injected in flow injection analysis mode. The solvent delivery system may be methanol/acetonitrile 50/50 (% v/v). Analytical HPLC may be performed with a Beckman System Gold using a reversed phase Waters NovaPack RP18 (5 μm) column (3.9 mm×150 mm), 254 nm detection, gradient 10-100% B (A=H₂O/0.1% TFA; B=CH₃CN/0.1% TFA) over 30 min, flow 1 mL/min. The products may be dried down in vacuo using a GeneVac evaporator system at 2 mbar.

μHPLC-MS/MS Analysis

Samples are resuspended in solvent, i.e. 50% MeOH/50% H2O/0.05% TFA. The samples are placed on a FAMOS μ-sampling workstation (LC Packings, Amsterdam, NL) and from there are loaded onto a column, i.e. an Inertsil ODS-3, C18, 5 μm, 50×0.8 mm column equipped with a C18, 2×0.8 mm μ-guard column (LC Packings). Chromatography is carried out using an HP1100 HPLC system (Hewlett-Packard, Waldbronn, Germany) running at 100 μl/min. The flow is split by an Acurate μ-flow processor (LC Packings) installed between the HPLC pump and the injector of the FAMOS workstation to yield a flow of 25 μl/min on the reversed-phase column. The HPLC solvents are:

A: 0.05% TFA/water

B: 96% acetonitrile/0.05% TFA/water.

Linear gradients from 10% to 80% B are run over 12 minutes, with an isocratic step of 3 min at 95% at the end of the gradient. After a 7 minutes re-equilibration step the next sample is injected. The column effluent passes through a U-Z view 30 nl flow cell (LC Packings) with the detector set at 214 nm, and from there is then directed through a fused silica capillary (340 μm o.d.×50 μm i.d.) into the elctrospray source of an LCQ ion trap mass spectrometer (Finnigan Corp., San Jose, Calif., USA) run with one 20 minutes segment composed of three scan events (1: MS mode for mass determination with the scan range set between 150 and 2000 amu; 2: dependent zoom scan mode with a minimum signal required of 100'000 counts; 3: dependent MS/MS scan mode for fragmentation of selected ions (highest ion above 100'000 counts) with the collision energy set at 32% and isolation width set at 7 amu). The sourc is operated at 4.5 kV with the heated capillary set at 220° C. and sheath nitrogen gas flow rate at 80. In the MS mode the ion time is set at 500 ms and the target number of ions at 5×10⁷; in the CID mode the ion time is at 500 ms and the target number of ions at 2×10⁷. In both modes 3 microscans/spectrum are performed. The electron multiplier is set at −1000 V and all spectra are collected in the positive-ion mode.

### FIGURES

FIG. 1/9 shows the HPLC-spur (UV) of the compound of example 14 (retention time: 11.38 min).

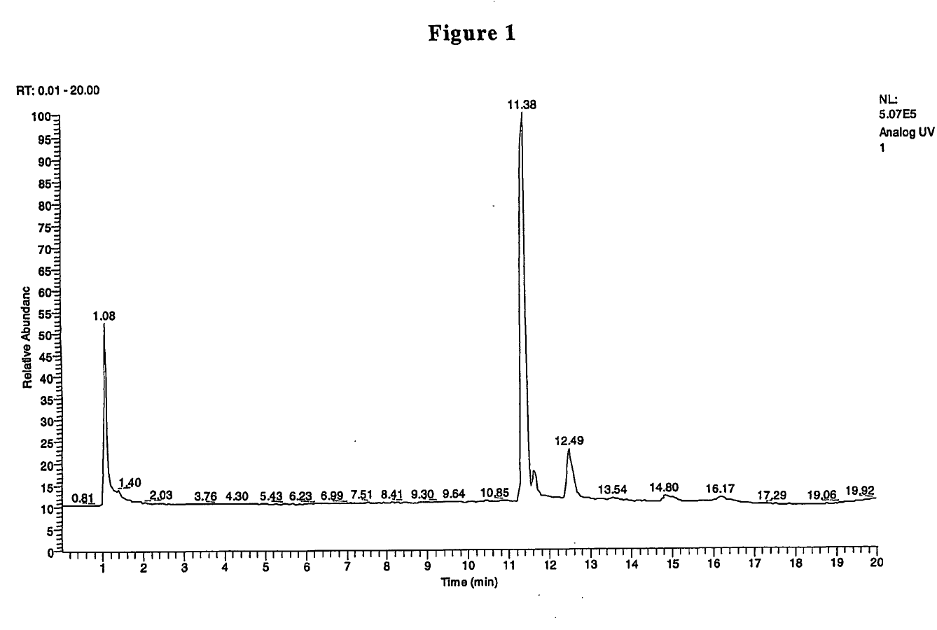

FIG. 2/9 shows the full MS of the compound of example 14 (range of retention time: 0.05-19.96 min).

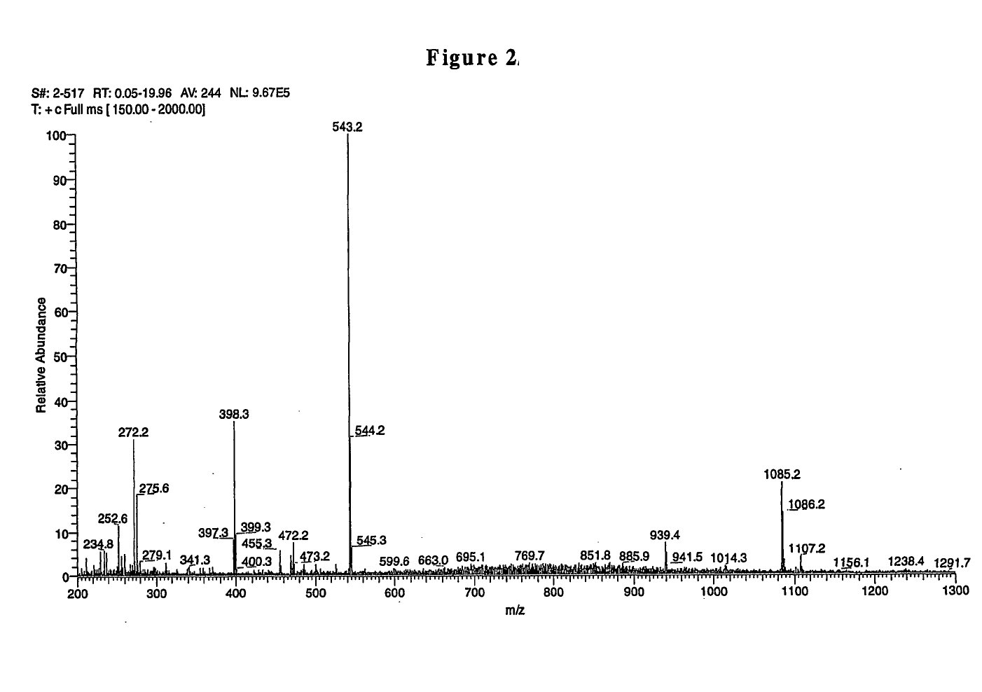

FIG. 3/9 shows the full MS of the compound of example 14 (range of retention time: 11.21-11.61 min).

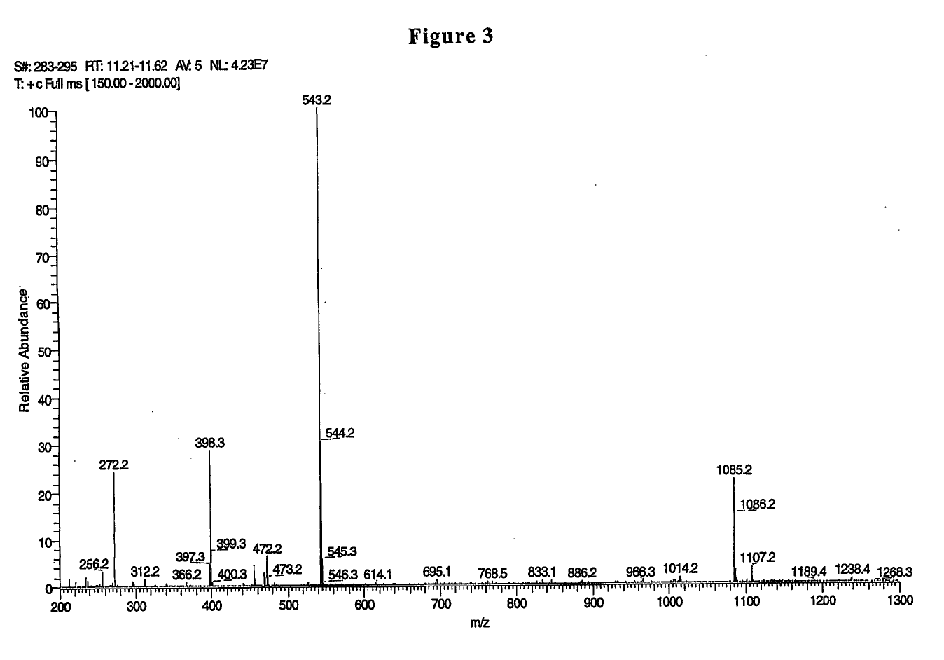

FIG. 4/9 shows the MS/MS of the molecular ion ([MH]⁺=543) of the compound of example 14 (retention time 11.61 min).

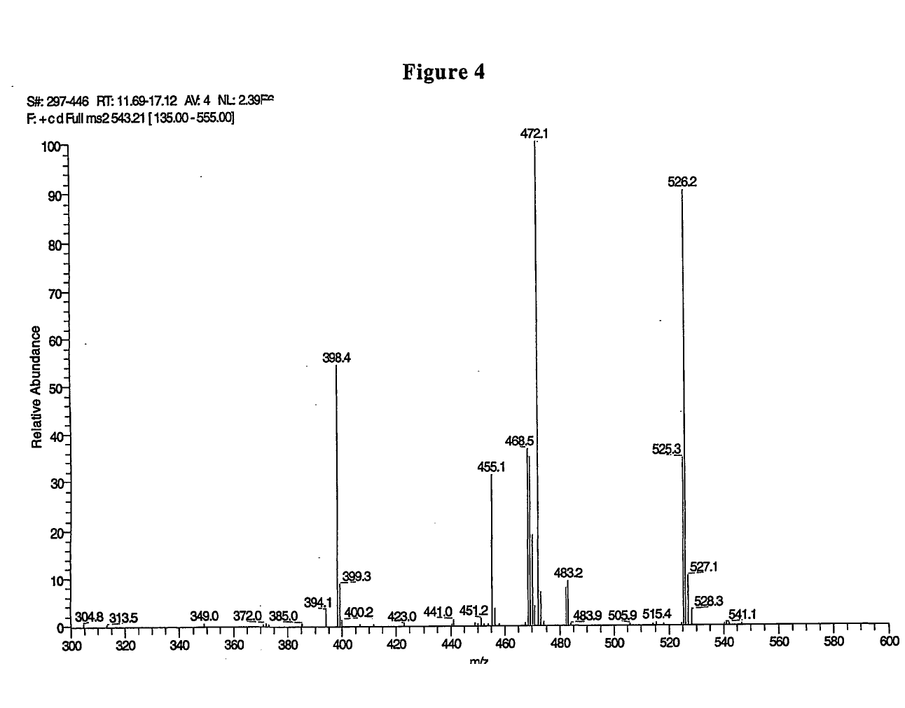

FIG. 5/9 shows the HPLC-spur (UV) of the compound of example 15 (retention time: 11.76 min; the compound at retention time 13.90 min is the trifluoroacetylated congener of compound of example 15 formed during photochemical cleavage in the presence of TFA).

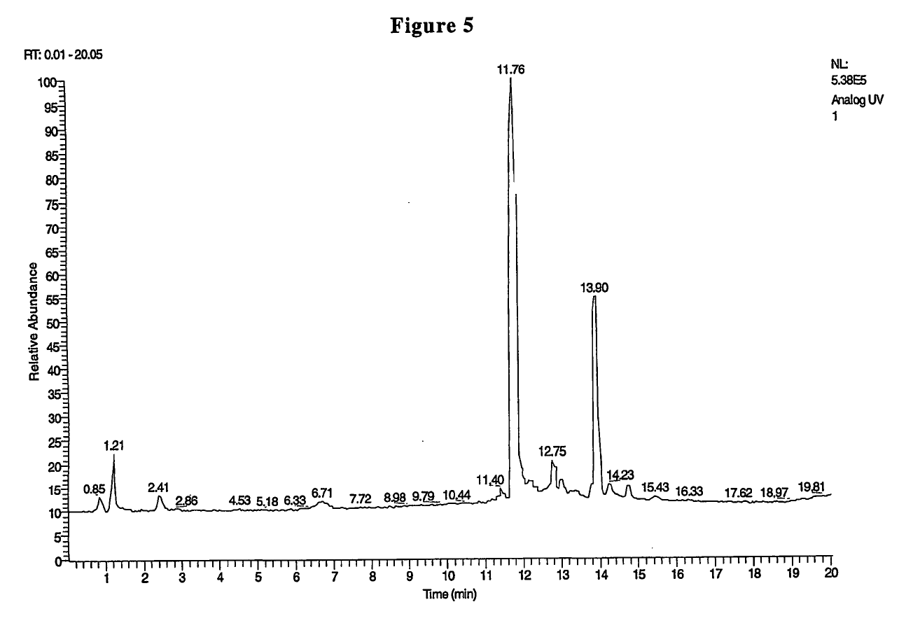

FIG. 6/9 shows the full MS of the compound of example 15 (range of retention time: 0.01-19.93 min).

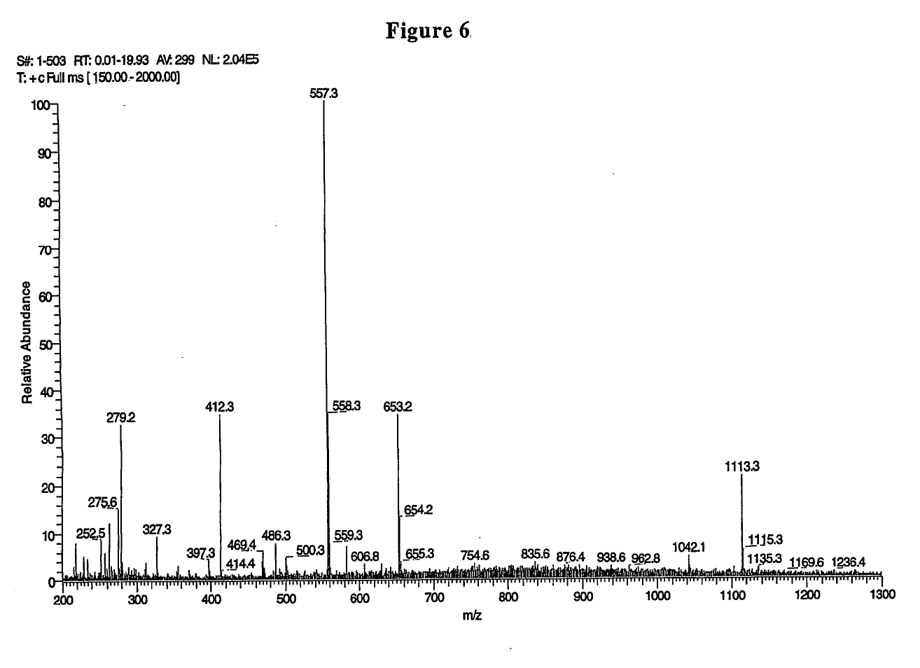

FIG. 7/9 shows the full MS of the compound of example 15 (range of retention time: 11.53-12.15 min).

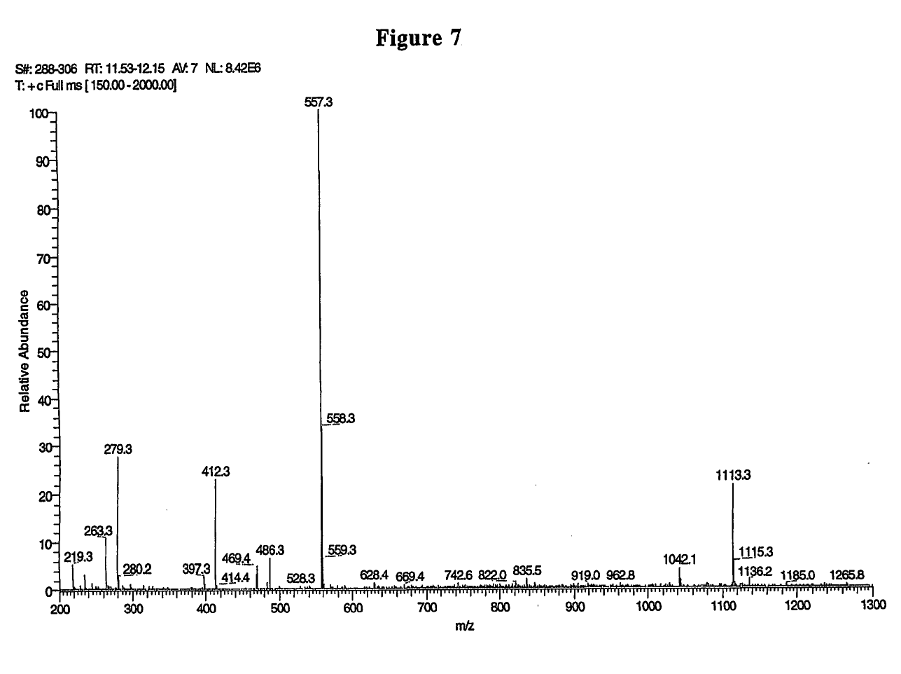

FIG. 8/9 shows the MS/MS of the molecular ion ([MH]⁺32 557) of the compound of example 15 (retention time 11.97 min).

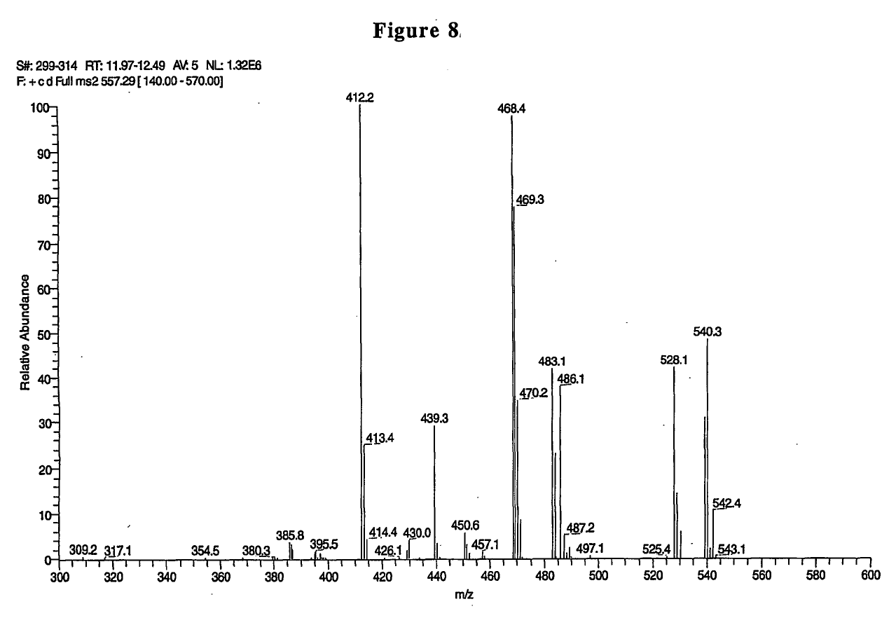

FIG. 9/9 shows the principle of on-bead screening with added AIDA-technology: Resin beads of a combinatorial compound library with tagged 1^{st} combinatorial step (R_{tag}; one-bead one-compound) are exposed to a target (e.g. a protein) labelled with a fluorescent dye. The excitation wavelength of the dye on the target is orthogonal to the AIDA-fluorophor (silent-AIDA) during inspection of the beads. Beads with bound labelled target are recovered (single bead-picking). The AIDA-R_{tag}-conjugated library compound (ligand) is cleaved from the single bead. Subsequently the on-bead binding event is confirmed and quantified in solution by determining the interaction of non-labelled target with the R-tag-AIDA conjugated compound by methods of fluorescence spectroscopy. Confirmed actives are submitted to single-bead LC/MS for decoding of the structure (MW and R_{tag}-reading) of the active ligand.

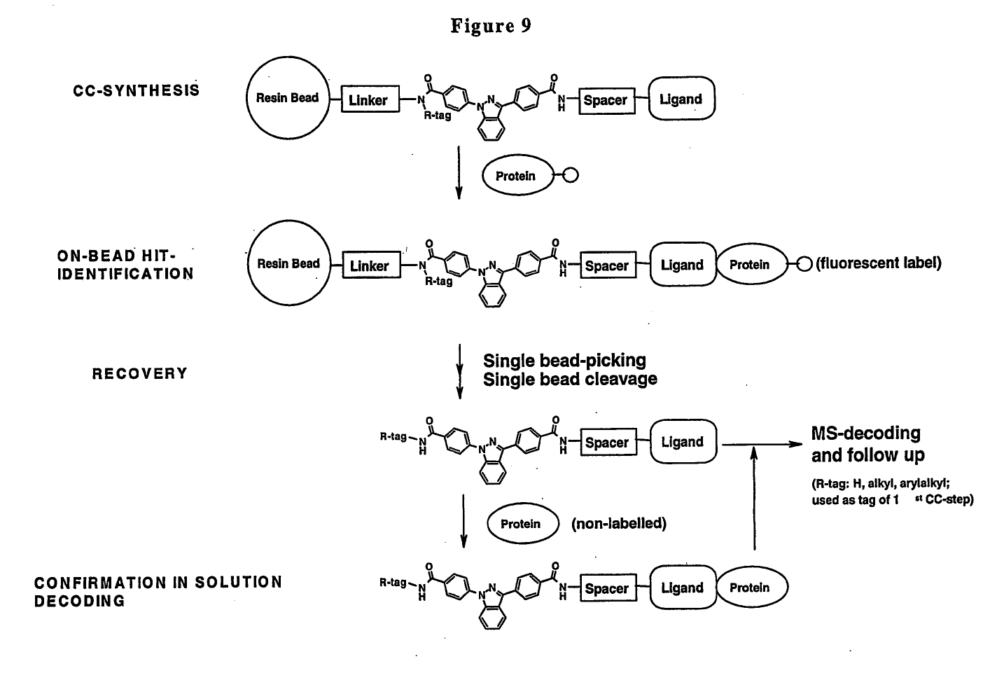

1. A compound of formula
A—B—D—X—D′—E II
or a compound of formula
A—B—D—E—D′—X II,
wherein
A is the residue of a solid support, originating from standard materials applied in solid phase and solution phase organic chemistry,
B is a linker residue having a group which allows cleavage of a compound of formula II or a compound of formula III to liberate an D—X—D′—E, or D—E—D′—X fragment, respectively,
D and D′ independently of each other are a bond or a spacer residue,
E is the residue of a molecule to be investigated produced via combinatorial chemistry, and
X is the residue of a fluorescent dye,
characterized in that a compound of formula II or of formula III is tagged by at least one tag residue.

2. A compound of formula
D—X—D′—E IV, or
D—E—D′—X V, or
E—D′—X VI
wherein E, and X are as defined in claim 1 and D and D′ independently of each other are the residue of a spacer, or are a bond, or are not present, characterized in that a compound of formula IV or of formula V or of formula VI is tagged by at least one tag residue.

3. A compound of formula I, wherein
one of R₁ and R₂ and one of R₃ and R₄ is hydrogen, and the other R₁ or R₂; and R₃ or R₄ is independently of each other
—COOH, —COOR₇, —CONHR_{tag}, —CONR_{tag} (CH₂)ₙOH, —CONR₈R₉, —CH₂OH, —CH₂NHR_{tag}, —NO₂, —NR₁₀R₁₁, —NR_{tag}COR₁₂, Cl, Br, F, —CF₃, unsubstituted alkoxy and alkoxy substituted by
-aryl, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂)ₙNRₜₐH,
unsubstituted alkyl or alkyl substituted by
—COOH, —COOR₇, —CONR_{tag}H, —CONR₈R₉, —CONR_{tag}(CH₂)ₙOH, —CH₂OH, —CH₂NH₂, —N═C═O, —N═C═S, —SO₃H, —SO₂NR_{tag}(CH₂)ₙNH₂, —SO₂NH(CH₂)ₙNR_{tag}H, —CONR_{tag}(CH₂)ₙNH₂, —CONH(CH₂)ₙNR_{tag}H;
R₅ and R₆ are hydrogen, or one of R₅ and R₆ is hydrogen and the other is hydrogen, halogen, unsubstituted alkoxy or alkoxy substituted by
-aryl, —NO₂, —NR₁₀R₁₁, —NR_{tag}COR₁₂;
unsubstituted alkyl or alkyl substituted by
—COOH, —COOR₇, —CONHR_{tag}, —CONR₈R₉, —CONR_{tag}(CH₂)ₙOH, —CH₂OH, —CH₂NHR_{tag}, —N═C═O, —N═C═S, —SO₃H, —SO₂NR_{tag}(CH₂)ₙNH₂, —SO₂NH(CH₂)ₙNHR_{tag}, —CONR_{tag}(CH₂)ₙNH₂, —CONH(CH₂)ₙNHR_{tag};
n=2 to 8,
R₇ is a carboxyl-protecting or carboxyl-activating group
R₈ and R₉ together with the nitrogen atom to which they are attached form heterocyclyl, with the proviso that piperazine is excluded;
R₁₀ and R₁₁ are independently of each other hydrogen or R_{tag},
R₁₂ is alkyl, aryl, aralkyl, unprotected or protected amino or halogen;
R_{tag} is hydrogen, alkyl, cycloalkyl, cycloalkylalkyl, aralkyl, heterocyclylalkyl, with the proviso that unsubstituted methyl and unsubstituted cyclopropylmethyl are excluded;
with the proviso that in a compound of formula I
at least one R_{tag} is present,
at least one R_{tag} is other than hydrogen, and
at least one functional groups are present in the meanings of R₁, R₂, R₃, R₄, R₅, and R₆ which have the ability to covalently bind to one or two further reactants.

4. Use of a compound of any one of claims 1 to 3 in the decoding step of a screening process.

5. A process for the synthesis of generic fluorescence labelled libraries (AIDA-libraries) on a conventionally used linker cleavable by light wherein the first combinatorial step of fluorescence labelled libraries is partial-encoded comprising providing a set of compounds bound to at least to on tag residue, which s t of compounds consists in compounds as claimed in any one of claims 1 to 3, wherein the tag residues have different molecular weights.

6. A process for the synthesis of generic fluorescence labelled libraries (AIDA-libraries) on a conventionally used linker cleavable by acid, wherein the first combinatorial step of fluorescence labelled libraries is partial-encoded, comprising providing a set of compounds of the present invention covalently bound to at least one tag residue which set of compounds consists in compounds as claimed in any one of claims 1 to 3, wherein the tag residues have different molecular weights.

7. A process for the decoding of building blocks of generic fluorescence labelled libraries (AIDA-libraries) comprising reading a tag residue in a compound of formula I, IV, V, or VI according to any one of claims 1 to 3 by use of MS-spectrography.

8. A process for the decoding of building blocks obtained by more than one combinatorial step of generic fluorescence labelled libraries (AIDA-libraries) comprising a split-and-mix-and-divide production of generically fluorescence (AIDA) labelled compound libraries in more than one combinatorial step and reading a tag residue, e.g. R_{tag}, in a compound of formula I, IV, V, or VI according to any one of claims 1 to 3 by use of MS-spectrography.

9. A process for the decoding of building blocks of generic fluorescence labelled libraries (AIDA-libraries) comprising
a) tagging a first combinatorial step with a tag residue and carrying out the first combinatorial step, in different vials for each building block expected to be obtained,
b) mixing the content of the vials containing the different building blocks obtained in the first combinatorial step,
c) carrying out a second combinatorial step such, that the building blocks obtained after the second combinatorial step have different molecular weights in a mixture obtained in b), for each building block in separate vials, and, if desired,
d) mixing the content of the vials obtained in step c) and carrying out a third combinatorial step, for each building block in different vials, and if desired,
e) carrying out a fourth combinatorial step for each building block in at least one of the vials obtained in step d), and
f) reading a tag residue in a compound of formula I, IV, V, or VI according to any one of claims 1 to 3 by use of MS-spectrography.

10. Use of a compound comprising a tag residue according to any one of claims 1 to 3 in the modification of physio-chemical properties of generic fluorescence labelled conjugated ligands.

11. Use according to claim 10 in the improvement of the solubility of generic fluorescence labelled conjugated ligands.
